(19) United States
(12) Patent Application Publication
(10) Pub. No.: US 2006/0068030 A1
(43) Pub. Date: Mar. 30, 2006

(54) Individual need-based system for providing supplements

(75) Inventors: James C Jensen, Henderson, NV (US); Herbert L Sugerman, Las Vegas, NV (US)

Correspondence Address: SANTANGELO LAW OFFICES, P.C., 125 SOUTH HOWES, THIRD FLOOR, FORT COLLINS, CO, 80521, US

(73) Assignee: Nutri-Check Technologies, LLC, Fort Collins, CO (US)

(21) Appl. No.: 10/541,621

(22) PCT Filed: Jan. 6, 2004
(86) PCT No.: PCT/US04/00179

§ 371 Date: Jul. 6, 2005

Related U.S. Application Data
(60) Provisional application No. 60/438,426, filed on Jan. 6, 2003.

(51) Int. Cl. B65D 83/04; A61K 33/10; B65D 85/42

(52) U.S. Cl. 424/687; 206/532

(57) ABSTRACT

A system for individual need-based determinations relative to the use of a substance is presented in various embodiments. As applied to supplements the system may be configured to present a test modality (6) which may be attached to a distribution container (1) within which supplements (2) or the like may be contained. The test modality (6) may be a user practical test modality that may be provided to a user with an information display (11) that may include an internal display (12) to educate the user on a particular test modality (6). Provisions for the options of a test result recordation enablement element (10) are provided in both manual and internet forms. The test modality (6) may be test strips compactly assembled and provided attached to the distribution container (1).

### TECHNICAL FIELD

This invention relates to systems for providing supplements such as nutritional or other supplements, and largely uncontrolled substances which individuals decide themselves to use for some type of human development or health benefit. It is specifically relates to the field of supplements such as vitamins and minerals but may also be applied to other substances which enhance either physical or mental development. Specifically, the invention relates in an initial application to providing calcium in a manner in which individuals are permitted to assess their own personal needs and are permitted to assess the effectiveness of the supplement

### BACKGROUND

The invention is most easily understood in the context of an initial application in the supplement industry. The supplement field represents a significant industry that provides many people considerable benefit. As most people already know, it is a fairly unregulated industry and often the supplements provided are not such that they cause great harm if misused. Thus, people are largely left to themselves to decide whether or not to use any particular supplement. While in some instances licensed medical practitioners may advise patients to use a particular supplement, many, many users of supplements make the decision themselves based upon advice from others, friends, articles, books, innuendo, and the like. Thus, whether or not to take a particular supplement and even the amounts taken can often be the result of an individual evaluating large group recommendations, making personalized estimates, and perhaps judgment calls made on inadequate information. This type of usage is dramatically different from many controlled substances such as prescription drugs and the like where the entire paradigm of use has developed from a more scientific basis. For example, a prescription drug is authorized by a trained, and often licensed, medical practitioner who preferably has specialized knowledge to individually determine and prescribe the propriety of and amount of use of a controlled substance for a particular person. The medical practitioner may even have available to them laboratory testing and other scientific ways of determining when a particular supplement is needed.

Unfortunately, to the typical supplement user usually none of these resources are utilized. Perhaps surprisingly, the entire test-diagnose-treat approach such as shown in U.S. Patent Publication Number U.S. Pat. No. 2,002,0065217 is not usually made available to the user of a supplement. While of course, any individual could actively seek some type of laboratory testing to determine the propriety of or even amount of a particular supplement both the propriety of a particular test and even the accessibility of such testing has not typically been facilitated for a user of a supplement. This is surprising because the entire test—prescribe paradigm is so well established for controlled substances that it might be expected that the mere uncontrolled nature of a particular substance would not necessitate a paradigm change. For example, it is common to use markers or indicia to qualitatively if not quantitatively evaluate many medical conditions and to assess the need for a particular substance. The existence of such markers for substances generally considered to be in the field of self administered supplements or the like, however, is not highly utilized. Rather, use is simply made based upon individual decisions. Often these decisions may be based on less than scientific criteria. For example, referring to U.S. Pat. No. 6,210,976 it can be understood that efforts have been undertaken to identify a particular marker for cardiac ischemia and them to prescribe the appropriate substance to treat such a condition. This is a common practice in the prescription medicine fields. Unlike this type of approach, however, the field involving the self use of supplements is not based on such quantitative or semi-quantitative indicators but often is merely left to personal decision. The present invention not only addresses this aspect, but it also provides system which can be applied to enhance the use of even controlled substances such as prescription drugs.

Beyond the simple paradigm shift apparent to some degree with respect to self administered supplements, there is a further aspect which is surprising. Even in areas where vitamin deficiencies have been the subject of scientific test criterion, the user has not usually been empowered to use such testing. For example, as explained in PCT publication WO00246746 tests have been developed for vitamin D evaluation. Such tests, however, are not largely user friendly and do not lend themselves to persons not having specialized medical or laboratory training—the typical users of supplements. Thus tests are often too complex and can be prohibitively complex for ordinary users. As shown in PCT publication WO08001415 a test for pancreatic function is highly complex. Similarly, the test for vitamin B12 deficiency as explained in PCT publication WO07900880 is one that involves a radioisotope complex and is thus not something that a typical user of supplements could easily implement. Thus both the complexity of the test and perhaps the risks of a false indication, as explained in U.S. Pat. No. 4,188,189, may have lead those in the field not to consider applying the more scientific paradigm to the supplement field. Test may have been viewed as too difficult or too dangerous to permit them to be made available to a typical user.

It is even surprising that a more scientific and user friendly approach has not developed for supplements because it has been known for years that the need and even absorbability of supplements can vary from person to person. For example U.S. Pat. No. 6,361,800 explains that vitamin needs and condition etiologies can vary significantly in humans. Thus it is surprising that while various tests have been known, they have largely not been made readily available to users of supplements in a manner that practically empowers to user to make sound decisions.

Testing is particularly appropriate where the supplement is a mineral such as calcium. Calcium is potentially one of the more important substances where deficiency can cause a variety of diseases. For instance, Robert Barefoot in “Barefoot on Coral Calcium: An Elixir of Life”, Wellness Publishing, Inc. 2001, explains the fact that calcium is a substance that not only is needed but can have its effectiveness varied by its very form or by the substances it is ingested with. Thus, calcium is a substance where its bioavailability, its need, and even its effectiveness can vary significantly from individual to individual. As noted in the article “Optimal Calcium Intake”, JAMA, Dec. 28, 1994, not only is calcium intake critical but conditions under which it is used, individuals who use it, and the environment can all dramatically vary the particular calcium needs of an individual. Unfortunately, even that article seems to lead away from an individualized regimen. As is traditional for many supplements, it seems to suggest screening populations and group-based, statistical determinations for calcium usage. Even U.S. Pat. No. 5,597,595 B—which recognizes that calcium needs can vary based on age and other individualized factors B—still applies an approach designed for the masses rather than for an individual. As yet another example, U.S. Pat. No. 5,817,351 discusses the individualistic nature of the need for calcium and yet still approaches its provision by a large population determined paradigm. Thus in spite of the fact that as recognized in the book by Rudolph Wiley entitled “Biobalance”, Essential Science Publishing, 5th printing 1998, that acidic versus alkaline diets can largely affect one's health and ones calcium needs, and in spite of the fact that the Barefoot reference comments about body pH, such criteria have not been made readily available to users of even the calcium supplement. Further, as explained in U.S. Pat. No. 4,520,112 in explaining a test for calcium presence, the entire test paradigm B that of radioactive assay B is not one typically made available to users and is not the type of test that a user would or could practically implement themselves to determine their individual need. Even tests such as that explained in U.S. Pat. No. 5,260,219 where test strips are disclosed, have not been associated with a supplement or other such substance at the point of sale or use so that a user can have the confidence and can be provided the convenience of a test modality known to be somewhat relevant to the use of the particular substance.

Thus, there has been a need for a system through which users and individuals making a personal decision relevant to the use of a supplement or other substance can be empowered at the time of purchase with sources upon which to base their decisions.

### DISCLOSURE OF INVENTION

The present invention can be configured in many different embodiments to provide a system through which individuals can make need-based decisions based on their own personal circumstance rather than on fitting within a statistical norm or the like. In an initial embodiment the invention involves a supplement provided within some sort of distribution container with an appropriate test modality physically attached to the container and provided with the supplement to the purchaser at the time of purchase. As applied to a calcium supplement, the test modality may be as simple as providing a stacked number of pH test strips to be able to both understand an initial need for and determine to some degree the effectiveness of a particular calcium supplement for that individual. These pH test strips may be physically attached to the distribution container and may even be provided free of charge so that the user can understand with more confidence their own individual circumstance. Thus when the user purchases the item of interest they may also receive with that item one or more test elements with the product. These test elements may be as simple as a saliva based pH test strip where the user is immediately informed of circumstances within their body so that they may have some indicia as to their need or even the effectiveness of a particular supplement or other substance. As a way of marketing the product, a degree of confidence in the product itself can be shown by providing the user and enabling the user to test themselves before, during, and after use of the particular product to assess it usefulness. This entire system of course is one that may be adapted to other substances and other test modalities as such are developed or applied.

Thus, it is a goal of the invention to provide a test modality with the distribution of the substance so as to enable or empower the purchaser to make individual, need-based decisions based on some type of evidence arguably relevant to the use of the substance rather than to merely use their own guess work in deciding upon a use. It also a goal of the invention to provide for an easy and user-practical test framework within which users may easily make decisions and have indications of their own personal circumstance. Yet another goal of the invention to provide for a generic approach that may be adapted as testing becomes more available and for an approach that may be applied to a variety of substances. Naturally other goals exist and some are explained throughout this specification.

### BRIEF DESCRIPTION OF THE DRAWINGS

FIG. 1 is a schematic diagram showing a distribution system with a test modality attached thereto.

FIG. 2 is a schematic showing the distribution system with the test modality merely associated with that distribution system at about the time of sale.

FIG. 3 shows an initial application of the invention as applied to a bottle and cap type of distribution system.

FIG. 4 shows one type of attachment system where a pH test strip is physically attached to a cap with a shrink wrap element.

FIG. 5 shows information and instruction aspects of a system as shown in FIG. 4.

FIG. 6 shows one example of a manual recordation system that may be provided as part of one embodiment.

FIG. 7 shows a schematic design of an Internet reporting system which may be used to monitor and assist the user in use of a particular substance.

FIG. 8 is an example of information that may be provided in one embodiment as well as potential ingredients for an embodiment.

### BEST MODES FOR CARRYING OUT THE INVENTION

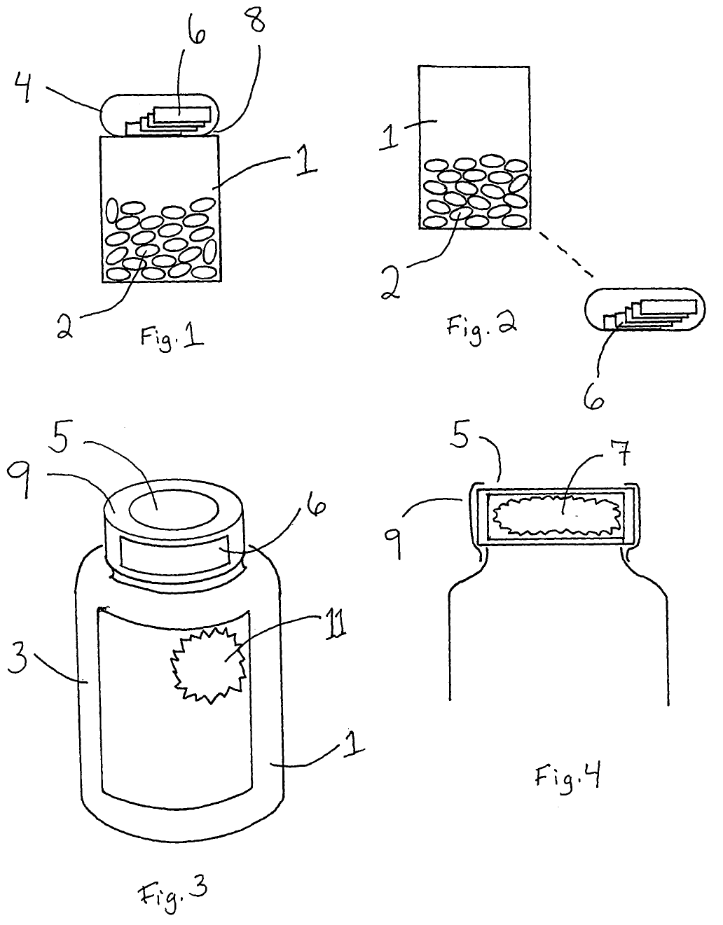

As can be understood from the discussion, the present invention may be embodied in a variety of ways. Although discussed in the context of a particular initial application such as that of the pH test strip-calcium system, it should be understood that the various elements can be altered and even replaced or omitted to permit application for other substances and other tests as appropriate. Referring to the figures beginning with FIG. 1, it can be seen that in one sense the invention involves a container within which a selected amount of some type of substance is contained. This supply quantity may be of a supplement such as calcium or may be of some other type of substance. The distribution container (1) may serve to contain or even to preserve the integrity of a particular supplement (2) or the like. As shown in FIG. 3, the distribution container (1) may be configured as a supplement bottle (3), essentially some type of enclosure within which a supplement is provided. The distribution container (1) may also have a closure (4) which may be removably connected to the distribution container (1). Again as shown in FIG. 3, this closure (4) may be as simple as a cap (5) on a supplement bottle (3). The cap (5) may be configured to be positioned on top of a distribution container such as a supplement bottle (3).

As mentioned earlier, the invention provides that some type of test modality (6) may somehow be associated with the supply quantity of the supplement (2). In one embodiment, it may even be important that the test modality (6) be associated with the supplement (2) at about the time the supplement (2) or other substance is made available for purchase. Further, it is even possible that the test modality (6) be associated with the actual distribution container (1). This is shown in FIG. 1 where the test modality (6) is schematically shown as attached to the distribution container (1) and by it being provided in association with some type of closure element (4) in that embodiment.

As illustrated graphically in FIG. 1, the test modality may be some type of test that has a quantity of disposable test items included. These items may be stacked so that a plurality of the test items is provided with the supplement (2). The test items may even be covered so that the quantity is more likely to be guaranteed to be associated with the supplement (2) at the time of distribution. As may be understood for test items that may be single use items or even disposable items, it is possible to provide a quantity of the test items where that quantity is coordinated with a particular quantity of the supplement (2) provided. Thus the user is not likely to be disappointed with a lack of test elements and similarly there is no waste such that the user is provided with more test items than are necessary. Thus it can be understood how the test itself may comprise a supplement supply quantity coordinated test.

As may have been understood from all the prior discussion, the type of test provided is desired to be a test that is useful to some aspect of use of the substance contained. When one understands that tests are often developed based upon the concept of markers or indicia which indicate the presence or absence of some analogue which lends itself to observation, it can be understood how the test itself be merely something that is arguably relevant to some aspect of use of the substance or perhaps the supplement (2). Further it may be understood that compromises may be made to balance the directness of a test determination with its ease of use. While ideally a spectrographic or radioisotope test may be preferred, it may be that a less precise, but more practical test be utilized. Of course, ideally the test modality may be one that is directly relevant to the use of the supplement or other substance. Similarly, merely something that highly correlates to the presence or absence of the supplement or an analogue may be used and thus it may be merely a high correlation test. This high correlation may be something that is statistically proven with perhaps varying degrees of uncertainty of result. Thus the statistical test may be one with a statistical P factor being anywhere from about 0.05 to 0.10 to 0.15. In situations where high correlation tests are not practical, it is possible or more practical to use a test modality that is merely indirectly indicative of the use of a particular substance. By using an indirectly relevant test, the user may be afforded at least some information even though it may not be the best type of information available. As mentioned, practicalities of course can come into play and it is possible that the expense or processes of a more thorough test is not justified in any particular instance. Thus, the indirectly relevant test may even be a test based upon publicly espoused test modalities or popularly used tests so that general familiarity may aid in the user having a better understanding of the test and its application. This may be important because it is a goal of the invention to provide a user-practical procedure or a user-practical test for the purchaser.

Further, in one aspect, such a test may be compactly assembled so that the user is provided a compactly assembled test modality with the supplement (2) or other substance at about the time of purchase or at about the time of accomplishing the step of providing the supplement or other substance to the user. In situations in which the test modality (6) is physically attached to the distribution container (1), the test modality (6) may or may not be provided in its own test container. The entire distribution container may also present a combined supplement and test container. In this fashion the supplement may be marketed in a more desirable manner such that the user is provided, perhaps even free of charge, an opportunity to test themselves as to whether they have a need or as to whether the actual supplement is providing the desired result. This marketing advantage can provide an enhanced experience for the user as well as can provide advantages to the distributor or manufacturer so that their product is selected over other similarly situated products because it provides more value for the user or purchaser.

As mentioned earlier, calcium is a supplement where the need is both highly individualistic and where a particularly practical test modality is now identified and available. Thus, by providing an amount of the calcium supplement with saliva pH test strips and even perhaps an explanation of a saliva pH test strip procedure and results interpretation, the user can more appropriately determine their own need and perhaps to some degree the effectiveness of a particular calcium supplement. Importantly, in this particular embodiment, the supplement (2) may be provided with the pH test strips attached to the actual distribution container (1). By so providing the user the test strips, the step of enabling the accomplishment of the test is effected. Thus, the element attaching the test strips to the distribution container (1) such as an attachment element (8), can serve as a test enablement element. In the initial embodiment where a pH test strip is provided in conjunction with a calcium supplement, the combined supplement and test container may provide both the supplement (2) as well as a test strip modality (6). This test strip modality (6) even be a plurality of saliva pH test strips which may be used for calcium as well as other substances. Thus by attaching or providing the saliva pH test strips to the distribution container (1), the distributor may enable the accomplishment of the test modality at the time the purchaser is provided the product.

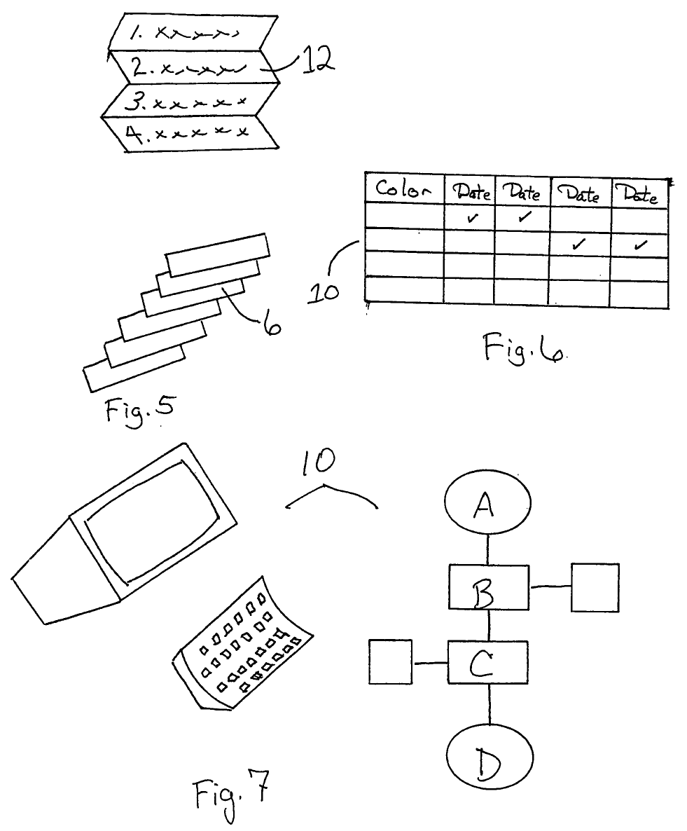

One aspect that can be important is the aspect of informing the user of both the presence of and use of the particular test modality (6) that may be attached to the distribution container (1). For example, an information display (7) may be provided to easily inform the user of this innovative advantage. Besides informing the user, it is also possible to even externally educate potential users so that they may understand the deterministic advantage that a particular test modality, such as a pH test strip modality, presents. This can be accomplished through an information display (7) as indicated in FIG. 4. This information display can highlight to a potential purchaser or user not only that the test modality (6) is provided with the supplement (2), but it may also educate the user, or potential user, as to the purpose and value of the test modality (6). This can be accomplished easily for tests such as the pH test strip modality explained earlier. Since pH test strips are easily used and are somewhat known, the instructions can very simply explain to a potential user both the value of and the ease of use of the particular test modality.

As mentioned earlier it is desired that the test modality (6) be somehow associated with the distribution container (1) at about the time that the purchase decision is made or at the time when the supplement (2) is actually provided to a user. This may be accomplished by physically associating the test modality (6) with the distribution container (1) such as is accomplished when the test modality (6) is actually attached to the distribution container (1). The test modality (6) may be exteriorly attached by some exterior attachment element (8). Physical association may be either direct, such as when attaching the test modality to the distribution container (1), or may be indirect where the test modality (6) is merely associated and highlighted to a purchaser as a separate product made available to the purchaser. This is shown schematically in FIG. 2. Referring back to FIG. 1, it can be seen that the test modality (6) may even be attached to the distribution container (1) through some type of exterior attachment element (8). An exterior attachment element (8) may attach the test modality (6) at some location, perhaps to the top of the distribution container (1) and as such may actually become a top attachment element.

An attachment element can also serve to connect the test modality (6) to the distribution container (1) and may also serve to conform or retain the test modality (6) in a conformed state with respect to the distribution container (1). As shown in FIG. 3, it can be seen in situations where the test modality (6) is conformed to the top of the distribution container (1), the exterior attachment element (8) may be some type of shrink wrap element (9). This can serve the advantage of combining a variety of functions for efficiency reasons. From one perspective, the shrink wrap element (9) may serve to conform the test modality (6) to the distribution container (1). It may also serve to seal the distribution container (1) and may also serve to attach the test modality (6) to the distribution container (1). Similarly, the shrink wrap element (9) may act as a tamper proof seal or tamper proof element through which the user can be assured that the supplement (2) within the distribution container (1) is provided in an unaltered state with quality assured. Since tamper proof seals are frequently used on supplements, the utilization of a tamper proof seal as the element to both conform and attach the test modality (6) can serve to aid in providing the test modality (6) at no cost increment to the end user or purchaser. Thus the user may receive a single combined supplement and test container that has a combined supplement and test container seal that may serve to attach the test modality such as the test strips to the distribution container (1). Naturally it should be understood that the test modality (6) may be attached to other items besides the distribution container as well and may even be provided in its own separate container. Thus a test modality (6) appropriate to some item even beyond a supplement or other substance may be accomplished in accordance with the present invention.

An aspect that may be important in providing a user-practical test is that the test modality (6) may be configured as a test modality that requires no separate equipment to accomplish the test. It may thus present a user-practical procedure so that the user can easily make a determination with respect to the use of the supplement (2) or other substance. In explaining that the test should be a user-practical test, it is also possible that the test be configured to be a largely error tolerant test such that misuses or mistakes in usage of the particular test can be tolerated without misleading or perhaps without dangerous results. Further, since users may be largely unskilled with respect to the test, it may be possible to utilize or configure the test so that it is a fail safe test modality so that in the event of misuse or mistake in usage, the test indication does not yield a result which might mislead the user toward a situation that could be dangerous for the user.

One aspect of providing a test that is practical for users can be the aspect of providing a test that yields immediate results. As such, the test modality (6) may actually serve as an immediate results test. This can also exist where the test is a test which provides results or is configured to provide results within about three, six, or even ten seconds, or more generally, an amount of time which a user is likely to tolerate and still continue to use and to value the test itself. Similarly, slower tests such as tests yielding results in about 20, 60 or even within about 300 seconds are possible. Importantly, both the procedures accomplished by the user and the actual time it takes for the user to discern results may be factored into the goal of providing a practical test that a user is likely to implement.

As shown in FIG. 1, the test modality (6) may be a plurality of compactly assembled test strips in some embodiments. The test may be disposable or reusable. Where compactly assembled, the test itself may practically take up no appreciable volume and may be provided as an easy addition at the time of purchase. The actual procedures used may also be selected so that the user can safely and assuredly use the test modality (6). For example, by configuring a test to require no more than about three substantive steps, a user may be able to quickly understand and use the test and may be able to accomplish the test himself or herself with a high degree of confidence. This test may be configured to have the user lick an item such as in a saliva based pH test strip or other such test, or may be configured for the user to dip or mix an item or substance such as in a test strip where immersion in some solution is desired. The test may serve to indicate a need for a supplement and as may actually serve as a supplement-need-indicative test or may even serve as a supplement-efficiency-based test where a user can understand their particular individual ability to absorb and assimilate a particular supplement or other substance.

As mentioned earlier, an enticement to the user at the time of sale may be that the test be provided as a no-cost increment to the purchase of the particular substance. By providing a no-cost increment test, the user may understand and may even be informed that the test is indicated as a free test at the time of purchase. This can enhance the marketing of a product and can expand the use, effectiveness, and value of a supplement for a user or purchaser.

It may also be important that the test results be made more accurate for any given situation. This may be accomplished by both configuring the test and/or instructing the user relative to certain types of test aspects. The test may be configured as a test which is profiled to a particular user. Thus, the test modality (6) may actually be a user-profiled test such as a test that is targeted toward women, children, men, or even particular age groups. Each such target may present a group within which a particular chemistry or condition is likely to exist which may then allow the test to more appropriately achieve its results. The target groups may even be conditional or prior habit groupings such as a target group of menstruating or menstruational women, a target group of post menopausal women, a target group of persons having particular habits such as coffee drinking, smoking, vegetarians, or the like. Enhanced and more accurate test results may also be achieved by having the user make decisions or provide input relative to their prior history or symptoms. It is even possible that typical results such as prior blood test results might be used to more appropriately gauge the type of test to be used. In a variety of embodiments, the test modality (6) may be configured for any such possibilities. Similarly, a personal baseline test might be accomplished from which changes off the baseline may be determined. As may be appreciated, a variety of options may be pursued so as to yield the appropriate, more accurate result.

As mentioned, not only may the test itself be configured for specific users, but the test instructions themselves might serve to reduce test variability. Either the test or the instructions may serve as a test variability reduction element. When instructions themselves are used, the instructions may direct the user to achieve an approximately constant parameter before or during the test procedure. For example, an approximately constant parameter test may be a test that directs the user to accomplish the test at a particular time of day that may be tightly controlled or recommended. The test may be a pre-meal test procedure, a particular meal type of test procedure (as might be appropriate when body acidity is being tested), a morning-based test procedure, an evening-based test procedure or even a multiple-times-a-day test procedure. For practical reasons and because the amount of personal variability may be tolerable, the test might be configured to serve as a weekly test procedure or a test procedure that is simply accomplished at a particular time after eating such as one hour- or three hours-after eating or the like. The test may also be configured to tie with body cycles such as menstrual cycles or other items such as circadian rhythms and the like. Each of these may provide analysis options as well and the feedback system mentioned later can be used to some advantage in these aspects.

In selecting the particular type of test to be used, a great variety of tests are possible. As mentioned earlier, the test may be directly or indirectly relevant to some aspect of use of the particular substance involved. In the initial embodiment, that of calcium and a pH test strip, the test itself is very easily implemented. Naturally, other test strips might be selected and thus any type of test strip-based test might be utilized if appropriate to a particular substance. A great variety of other test modalities may also be used included, but not limited to, the following list: pH indicative substance, test strip-based test, chip-based test, culture-based test, absorption-based test, chromatagraphy-based test, antibody-based test, dye-based test, blood thinner-based test, vasodilator-based test, AIDS-based test, hormone-based test, a hormone replacement therapy test, temperature-based test, temperature strip-based test, thermography strip test, peripheral circulation-based test, user extremity-based test, red blood cell-based test, blood presence-based test, electrical conductivity-based test, skin electrical conductivity-based test, galvanic skin response-based test, magnetic response-based test, magnetic field-based test, electrical field-based test, electrical current-based test, color coded results-based test, testosterone-based test, absorption-based test, dipstick-type test, vaginal fluid-based test, sexually transmitted disease-based test, enzyme linked immuno serum assay-based test, kirlian photography-based test, reaction time-based test, photoelectric stimulus-based test, alcohol presence-based test, breath-based test, blood-based test, enzyme-based test, virus-based test, hormone-based test, fertility-based test, sperm motility-based test, sperm count-based test, viral byproduct-based test, neuramidase-based test, candida-based test, PCR-based test, saliva-sensitive test, urine-based test, hair-based test, nail-based test, non-invasive test, blood-based test, pH-based test, ketone-based test, urea-based test, serum albumin-based test, hormone-based test, immunoassay, enzymatic assay, free radical-based test, redox-based test, oxidative metabolite-based test, IgG-based test, IgA-based test, IgM-based test, venous plasma pH-based test, arterial pH-based test, free radical-based test, antioxidant-based test, chemical reaction-based test, NPN-based test, PNP-based test, a single parameter test, multiple parameter test, two stage test, three stage test, a self-contained test, a no more than three substantive step test, a user mouth-based test, and a user dip-based test.

As with the great variability of the particular type of test to be used, similarly the type of substance involved may vary greatly. As mentioned earlier in one embodiment the invention can be tailored to non-prescription substances and may even be tailored to supplements. These represent but one example and of course the invention may be applied to other substances as well. In applying the system to particular substances, it may be appropriate to select high value or high user discrimination substances initially. As mentioned earlier calcium is a type of substance not only because of its variability individually, but also because it is a type of substance where users recognize the variation among particular species of calcium. For example as mentioned in the article “Safety of Some Calcium Supplements Questioned”, there may be distinctions between refined calcium such as calcium carbonate as opposed to chelated calcium or a naturally occurring form known as coral calcium. Users who are likely to be well enough informed to discriminate between these types of supplements may also be likely to value a test modality that informs them of both the effects and need for a particular substance. As explained in the article mentioned earlier entitled “Barefoot on Calcium”, coral calcium may be a vastly different substance and may provide significantly enhanced health benefits as opposed to other forms of calcium. The distinctions in use by user grouping as presented in the article “Calcium the Facts” by Beth Ley, BL Productions 2001, may be important factors which may be utilized in presenting the system to users and in more accurately determining the type of test to be used and the implications of the results. The fossilized coral calcium referenced in that book may be a particularly appropriate substance to be tied to the present invention because users likely to distinguish at this level are well informed and will make good use of the attached test modality. In understanding the potential chemical makeup of a coral calcium, the elemental analysis presented in the article by B. W. Halstead entitled “Fossil Stony Coral Minerals and Their Nutritional Application”, World Life Research Institute, 1999, is incorporated by reference as well. Though the focus on coral calcium certainly helps in understanding the invention, it should be understood that the present invention is not at all limited to only a coral calcium supplement. Rather the invention is much broader and may be provided with a host of different supplements and other substances including but not limited to the following: vitamin, vitamin A, a B vitamin, vitamin C, vitamin D, vitamin K, mineral, calcium, potassium, sodium, coral calcium, calcium carbonate, chelated calcium, herb, ginko, ginko biloba, ginger, mandrake root, ayurvedic herb, native American herb, Chinese herb, Russian Herb, Japanese herb, ayurvedic medicinal herb, native American medicinal herb, Chinese medicinal herb, Russian medicinal herb, Japanese medicinal herb, ayurvedic supplement herb, native American supplement herb, Chinese supplement herb, Russian supplement herb, Japanese supplement herb, weight, loss substance, anorexia mitigation substance, metabolism increase substance, fat blockers (diet pills) can lead to acidosis, decreased fat absorption substance, herbal weight, loss substance, physical development substance, physical training substance, body building substance, nootropic substance, cognitive capability increase substance, memory capability increase substance, memory enhancement substance, sexual capability increase substance, depression mitigation substances, acetopyphin, ibuprophin, aspirin, test substance, AIDS test substance, and RU486. Embodiments of he invention may also be applied to substances which are designed to enhance desired traits such as body building substances, weight loss substances, sexual development substances, and any other type of mental or physical development substance. It may also be applied to prescription drugs and other regulated substances, even if the higher correlations required and potential danger of use of the particular substance may dictate the use of more highly correlated tests.

As mentioned earlier, the test itself can vary and may also present a test which is either directly or even indirectly relevant to some aspect of use of the particular substance involved. In one embodiment the test may present simply a qualitative test modality. It may also present a semi-quantitative test modality or may even be fully configured to provide a quantitative test modality. In instances where the absolute value is relevant, an absolute value test or absolute value test modality may be used similarly in instances where only a relative value is meaningful, a relative value test modality may be used. This may even be combined with some type of personal baseline test result so that constant comparisons can be made to show progress. Even a rate of change test modality may be used. Similarly, other substances and other tests may be presented even beyond a supplement context ranging from sexually transmitted disease tests to multiple regimen tests, that is tests where more than one result is combined to achieve more accurate results.

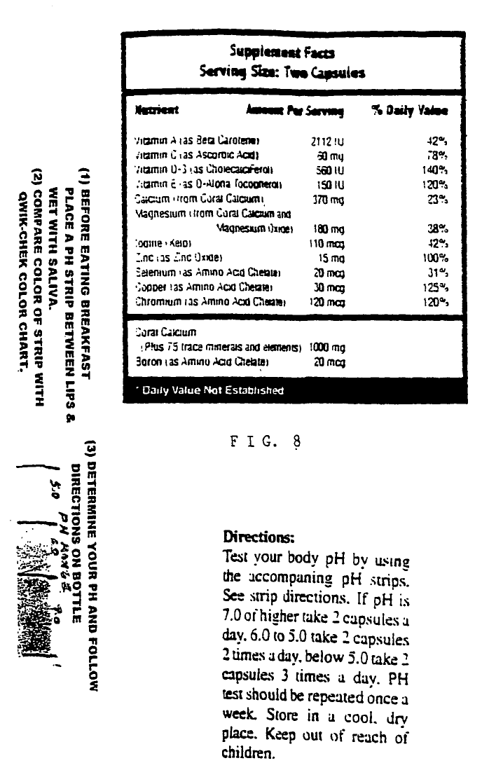

With respect to some test modalities, it may be valuable to record results of tests and perhaps even to report those results for tracking or analysis. In some embodiments, the invention may be configured to empower users to track the test results perhaps through some type of test result recordation enablement element. As shown in FIGS. 6 and 7, this test result recordation enablement element (10) may be a manual recording system or manual test result recordation element or an internet-based recording system or internet-based test result recordation element. Thus the user may be provided an option of recording their test results to track changes or perhaps enable separate analysis of the test results. As shown in FIG. 7, the internet-based test result recordation element may combine an internet data transfer whereby test results can be provided to a central facility and analysis of some sort or assessment can be accomplished. The internet procedure may accomplish accepting the user information (A), storing and comparing the information to other information for that particular user (B), analyzing that information in the context of both that individual user's information as well as other statistical information (C) and then even perhaps reporting back to the user some type of information ranging from statistical comparisons to individual concerns (D). Again, these should be understood as exemplary and not limiting as many other steps or functions may be provided in keeping with the invention.

The invention may also be configured to provide a system through individual serial numbers or private access codes whereby a user is provided access to an online reporting and analysis capability as part of the system or for an additional fee. This capability may accept the results entered and sent by the user and may automatically or manually accomplish an analysis of those results. In any situation where the results seem to yield an unusual indication, it is possible that automatic or manually intervened analysis can be accomplished and the user could be communicated some type of information. This information could be as simple as a potential error indicia and as such the internet system could serve as a potential test error indicia assessment system, or it could be simply a user feedback system. The system might also afford an opportunity to gather information and achieve more efficacious results for all users or for the distributing or manufacturing entity. In order to encourage users to report results, it is even possible that the system could entice the use of the internet-based recording system by providing discounts or repeat user benefits so that reporting results might yield benefits to the user and also might encourage constant use of the system. Further, by tracking potential errors and providing a user feedback system, the invention might provide additional benefits to users.

Another aspect of some embodiments of the invention is the fact that they may include an information display (11) to provide information a potential user. The information display (11) may be configured to provide a host of different types of information to a potential user. This information may range from information indicating the existence of the test, the value of the test, the cost of the test, or even how simple the test is to use. By providing a test use information display attached to the distribution container (1), the information display (11) may quickly indicate to a potential purchaser that the test is extremely easy to accomplish. By informing a potential purchaser of the existence of the test prior to about the time of purchase, the potential purchaser may realize and be able to distinguish from a supplement having the associated test and one not providing such a test. By the information display (11) being attached to the distribution container (1) the availability of the test may be temporally associated with the purchase of the product. As such, the test itself may be a temporally associated with distribution test. As mentioned earlier, the test may be physically associated with the product and so the test may be a physically associated with distribution test. On-site testing, that is testing accomplished at the time of purchase, or even send-in testing are also options that are possible. Importantly, in order to educate potential purchasers it may be important to externally display information on the product itself. This external display might indicate simplified procedures for use of the test as well as might generally instruct the user so that they may understand the value of the test associated with the product. By using the information display (11) to educate potential users, the information displayed may itself serve as a test education element. Importantly, in order to provide users with enough information to make quick decisions, it may be appropriate for the information display (11) to indicate or demonstrate perhaps a single step for the use of the test or perhaps at least not more than three steps for the use of the test. The information display (11) may also demonstrate the practical nature of the test visually or through text.

The entire information display may also include an internal detailed display such as is shown in FIGS. 5 and 8. The internal display (12) may be provided with the supplement (2) and with the test modality (6). It may serve to explain a test process that is useful to use of the test modality (6). As shown in FIG. 5, the internal display (12) may simply be printing inside a folded piece of paper within which the test modality (6) such as pH test strips may be contained.

As can be easily understood from the foregoing, the basic concepts of the present invention may be embodied in a variety of ways. It involves both distribution techniques as well as devices to accomplish the appropriate product distribution. In this application, the distribution techniques are disclosed as part of the results shown to be achieved by the various apparatus described and as steps that are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure. The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. It should also be understood that a variety of changes may be made without departing from the essence of the invention. Such changes are also implicitly included in the description and its application to a variety of substances and tests. They still fall within the scope of this invention.

Further, each of the various elements and steps of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “container” should be understood to encompass disclosure of the act of “containing”—whether explicitly discussed or not—and, conversely, were there effectively disclosure of the act of “containing”, such a disclosure should be understood to encompass disclosure of a “container” and even a. “means for containing.” Such changes and alternative terms are to be understood to be explicitly included in the description.

I. U.S. Patent Documents

| | | | | | FILING |
|---|---|---|---|---|---|
| DOCUMENT NO | DATE | NAME | CLASS | SUBCLASS | DATE |
| 3,941,565 | 3/2/1976 | Schwartz | 23 | 230 | 1/2/1973 |
| 4,039,080 | 8/2/1977 | Cappuccilli | 206 | 534 | 3/23/1976 |
| 4,147,514 | 4/3/1979 | Magers et al. | 23 | 230 | 11/21/1977 |
| 4,188,189 | 2/12/1980 | Allen | 23 | 230.3 | 4/4/1978 |
| 4,351,822 | 9/28/1982 | Allen | 424 | 1 | 8/24/1979 |
| 4,520,112 | 5/28/1985 | Snyder et al. | 436 | 504 | 3/9/1983 |
| 4,786,510 | 11/22/1988 | Nakel et al. | 426 | 74 | 7/2/1987 |
| 4,890,741 | 1/2/1990 | Edelstein | 206 | 534 | 8/17/1988 |
| 4,992,282 | 2/12/1991 | Mehansho et al. | 426 | 72 | 5/8/1989 |
| 5,064,071 | 11/12/1991 | Kerfoot, Jr. | 206 | 534 | 5/5/1990 |
| 5,071,769 | 12/10/1991 | Kundu et al. | 436 | 128 | 12/17/1987 |
| 5,108,767 | 4/28/1992 | Mulchandani et al. | 426 | 72 | 6/10/1991 |
| 5,199,877 | 4/6/1993 | Page | 434 | 262 | 6/14/1991 |
| 5,260,219 | 11/9/1993 | Fritz | 436 | 71 | 8/28/1991 |
| 5,597,595 | 1/28/1997 | DeWille et al. | 426 | 74 | 4/7/1995 |
| 5,642,837 | 7/1/1997 | Hays et al. | 221 | 197 | 6/30/1995 |
| 5,656,502 | 8/12/1997 | MacKay et al. | 436 | 180 | 6/7/1995 |
| 5,817,351 | 10/6/1998 | DeWille et al. | 426 | 74 | 1/27/1997 |
| 6,040,333 | 3/21/2000 | Jackson | 514 | 456 | 6/12/1997 |
| 6,080,431 | 6/27/2000 | Andon et al. | 424 | 602 | 1/6/1995 |
| 6,106,874 | 8/22/2000 | Liebrecht et al. | 426 | 74 | 11/18/1998 |
| 6,210,976 | 4/3/2001 | Sabbadini | 436 | 518 | 5/22/1998 |
| 6,361,800 | 3/26/2002 | Cooper et al. | 424 | 630 | 4/13/2000 |
| 6,383,751 | 5/7/2002 | Barendse | 435 | 6 | 10/23/1998 |
| 6,423,354 | 7/23/2002 | Monte | 426 | 72 | 9/1/2000 |
| 6,444,435 | 9/3/2002 | Christner et al. | 435 | 14 | 11/30/2000 |
| 6,458,547 | 10/1/2002 | Bryan et al. | 435 | 7.1 | 12/12/1997 |
| 6,461,875 | 10/8/2002 | Bar-Or et al. | 436 | 536 | 10/2/1998 |
| 6,475,743 | 11/5/2002 | Bar-Or et al. | 435 | 7.1 | 10/2/1998 |
| D408,128 | 4/20/1999 | Sheiner | D3 | 203 | 12/2/1997 |

II. Foreign Patent Documents

| DOCUMENT NO | DATE | COUNTRY | CLASS | SUBCLASS |
|---|---|---|---|---|
| AU0742109B2 | 6/22/1998 | Australia | C08K | 5/15 |
| BG0104878A | 10/23/2000 | Bulgaria | G01N | 33/00 |
| EP 1211512A2 | 11/23/2001 | Europe | G01N | 33/52 |
| GB 2223215A | 7/21/1988 | UK | B65D | 1/02 |
| JP20011192084A2 | 1/7/2000 | Japan | B65D | 85/72 |
| JP3081230A2 | 8/22/1989 | Japan | A61K | 49/00 |
| JP4320696A2 | 4/22/1991 | Japan | C12Q | 1/26 |
| JP4363659A2 | 6/10/1991 | Japan | G01N | 33/53 |
| JP58011815A2 | 7/14/1981 | Japan | G01G | 11/00 |
| JP58186050A2 | 4/23/1982 | Japan | G01N | 33/82 |
| JP59005956A2 | 7/1/1982 | Japan | G01N | 31/22 |
| JP6078795A2 | 9/7/1992 | Japan | C12Q | 1/26 |
| JP63148957A2 | 12/11/1986 | JP | A23L | 1/304 |
| JP7077528A2 | 8/3/1994 | Japan | G01N | 33/66 |
| JP7132065A2 | 11/9/1993 | Japan | A23L | 1/304 |
| JP8015211A2 | 6/28/1994 | Japan | G01N | 27/327 |
| JP8155470A2 | 12/5/1994 | Japan | C02F | 1/68 |
| NZ0504037A | 10/23/1998 | New Zealand | C12Q | 1/68 |
| NZ0504037A | 10/23/1998 | New Zealand | C12Q | 1/68 |
| SU1239594A1 | 4/6/1984 | USSR | G01N | 33/48 |
| WO 02/46746A2 | 12/6/2000 | PCT | G01N | 33/48 |
| WO 79/00880 | 4/4/1978 | PCT | G01N | 33/16 |
| WO 80/01415 | 1/2/1979 | PCT | G01N | 33/50 |
| WO 92/19251 | 5/6/1991 | PCT | A61K | 33/06 |
| WO 92/21355 | 5/28/1991 | PCT | A61K | 33/06 |
| WO 92/22218 | 6/10/1991 | PCT | A23L | 1/302 |
| WO 98/33488 | 1/31/1997 | PCT | A61K | 9/20 |
| WO 99/03921 | 7/18/1997 | PCT | C08K | 5/15 |
| WO 99/23248 | 10/30/1997 | PCT | C12Q | 1/68 |
| WO 99/56563 | 4/30/1999 | PCT | A23L | 1/29 |

II. Other Documents (Including Author, Title, Date, Pertinent Pages, Etc.)

CALCIUM: The Facts, A Health Learning Handbook, 1964, Beth M.
Ley, Ph.D., especially pp. 25
BIOBALANCE, The Acid/Alkaline Solution to the Food-Mood-Health
Puzzle, July 1989, Rudolf A. Wiley, Ph.D., especially pp. 7-18,
133-148, 198
Barefoot on Coral Calcium: An Elixir of Life?, Health Secrets of
Coral of Okinawa, 1940, Robert R. Barefoot, especially pp. 61-
114, 125-129 (www.wellnesspublishing.com)
The Acid Alkaline Balance Diet, An Innovation Program for Ridding
your Body of Acidic Wastes, 2002, Felicia Drury Kliment,
especially pp. 1-6
Optimal Calcium Intake, NIH Consensus Conference; Dec. 28, 1994,
Vol 272, No. 24, pp 1942-1948
Professional Products Information; Warren-Teed Pharmaceuticals,
Inc.; CAL-O-B ®; pp 1015
Safety of Some Calcium Supplements Questioned; Calcium
Supplements; Brief Critical Reviews, March 1994, pp 95-97
US 2001/0051173 Al, Nabi, et al., Published date Dec. 13, 2001,
Filed date Jul. 10, 2001.
US 2002/0065217 Al, Qian, et al., Published date May 30, 2002,
Filed date Aug. 04, 2001.
US 2002/0110844 Al, Christner, et al., Published Aug. 05, 2001,
Filed date Nov. 30, 2000.

All patents, publications, or other references mentioned in this application for patent or listed in the above listing are hereby incorporated by reference. The priority case, U.S. Provisional Patent Application No. 60/438,426, filed Jan. 6, 2003, is hereby incorporated by reference including any figures or attachments. In addition, as to each term used it should be understood that unless its utilization in this application is inconsistent with such interpretation, common dictionary definitions should be understood as incorporated for each term and all definitions, alternative terms, and synonyms such as contained in the Random House Webster's Unabridged Dictionary, second edition are hereby incorporated by reference. Finally, as to all references listed or specifically mentioned, each is hereby appended and hereby incorporated by reference, however, as to each of the above, to the extent that such information or statements incorporated by reference might be considered inconsistent with the patenting of this/these invention(s) such statements are expressly not to be considered as made by the applicant(s).

Thus, the applicants should be understood to claim at least: i) each of the substance distribution devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, and ix) methods and apparatuses substantially as described hereinbefore and with reference to any of the accompanying examples, x) the various combinations and permutations of each of the elements disclosed, and xi) each potentially dependent claim or concept as a dependency on each and every one of the independent claims or concepts presented. In this regard it should be understood that for practical reasons and so as to avoid adding potentially hundreds of claims, the applicant may eventually present claims with initial dependencies only or as Markush groupings. Support should be understood to exist to the degree required under new matter laws—including but not limited to European Patent Convention Article 123(2) and United States Patent Law 35 USC 132 or other such laws—to permit the addition of any of the various dependencies or separate claims or other elements presented under one independent or dependent claim or concept as dependencies or elements under any other independent claim or concept. Further, when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

In addition, the claims set forth later in this specification by are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1-9. (canceled)

10. A method of providing a supplement comprising the steps of:
containing an amount of calcium supplement in a distribution container;
selecting a plurality of saliva pH test strips useful to use of said calcium supplement;
establishing a saliva pH test strip procedure;
compactly assembling said plurality of saliva pH test strips;
attaching said compactly assembled plurality of saliva pH test strips to said distribution container;
externally displaying the presence of said plurality of saliva pH test strips to potential purchasers of said calcium supplement;
providing said amount of said calcium supplement to a purchaser; and
providing said compactly assembled plurality of saliva pH test strips to said purchaser of said calcium supplement at about the time of accomplishing said step of providing said amount of said calcium supplement to a purchaser.

11. A method of providing a supplement as described in claim 10 wherein said step of attaching said compactly assembled plurality of saliva pH test strips to said distribution container comprises the step of sealing said compactly assembled plurality of saliva pH test strips to said distribution container.

12. A method of providing a supplement as described in claim 1 and further comprising the step of demonstratively minimizing the likelihood of tampering of said distribution container by use of a tamper-proof seal, and wherein said step of sealing said compactly assembled plurality of saliva pH test strips to said distribution container comprises the step of utilizing said tamper-proof seal.

13. A method of providing a supplement as described in claim 10 wherein said step of providing said amount of said calcium supplement to a purchaser comprises the step of providing an amount of coral calcium supplement to a purchaser.

14. A method of providing a supplement as described in claim 10 wherein said step of containing an amount of calcium supplement in a distribution container comprises the step of containing an amount of calcium supplement in a distribution container having a supplement bottle and a cap on top of said supplement bottle.

15. A method as described in claim 14 wherein said step of attaching said compactly assembled plurality of saliva pH test strips to said distribution container comprises the step of attaching said test to said top of said distribution container.

16. A method as described in claim 15 wherein said step of attaching said test to said top of said distribution container comprises the step of shrink wrap attaching said test to said top of said distribution container.

17. A method as described in claim 14 wherein said distribution container has a tamper proof element, and wherein said step of attaching said test to said distribution container comprises the step of utilizing said tamper proof element of said distribution container.

18. A method of making a supplement available comprising the steps of:
containing an amount of said supplement by a distribution container;
selecting a test modality arguably relevant to some aspect of use of said supplement;
establishing a user-practical procedure for use of said test modality;
compactly assembling said test modality; and
providing said compactly assembled test modality for distribution in association with said distribution container.

19. A method of making a supplement available as described in claim 18 and further comprising the step of providing said compactly assembled test modality to said purchaser of said amount of said supplement at about the time of accomplishing said step of providing said amount of said supplement to a purchaser.

20. A method of making a supplement available as described in claim 19 wherein said step of providing said compactly assembled test modality to said purchaser of said amount of said supplement at about the time of accomplishing said step of providing said amount of said supplement to a purchaser comprises the step of attaching said compactly assembled test modality to said distribution container.

21-42. (canceled)

43. A supplement distribution system comprising:
a selected amount of said supplement;
a distribution container within which said selected amount of said supplement is contained; and
a compactly assembled plurality of test strips attached to said distribution container.

44. A supplement distribution system as described in claim 43 wherein said compactly assembled plurality of test strips comprise a plurality of compactly assembled pH test strips.

45. A supplement distribution system as described in claim 43 and further comprising an information display associated with said distribution container within which selected amount of said supplement is contained.

46. A method as described in claim 18 wherein said step of selecting a test to associate with said supplement comprises the step of utilizing a user practical test.

47-53. (canceled)

54. A method as described in claim 46 and further comprising the step of establishing said test as a no-cost increment to purchase of said supplement.

55. A method as described in claim 54 wherein said step of establishing said test as a no-cost increment to purchase of said supplement comprises the steps of:
informing a potential purchaser of the existence of said test prior to the time of purchase of said supplement; and
indicating said test as free at about the time of sale of said supplement.

56. A method as described in claim 46 and further comprising the steps of:
informing a potential purchaser of the existence of said test prior to the time of purchase of said supplement; and
demonstrating the practical nature of said test to said potential purchaser.

57. A method as described in claim 46 wherein said step of selecting said test comprises the step of utilizing a multiple test regimen.

58. A method as described in claim 57 wherein said step of utilizing a multiple test regimen comprises the step of indicating a frequency for said user to accomplish said multiple test regimen.

59. A method as described in claim 57 wherein said step of utilizing a multiple test regimen comprises the step of utilizing a temporally sequenced testing process.

60. A method as described in claim 46 and further comprising the step of empowering a test recording option.

61. A method as described in claim 60 wherein said step of empowering a test recording option comprises the step of providing a manual recording system.

62. A method as described in claim 60 wherein said step of empowering a test recording option comprises the step of providing an internet-based recording system.

63. A method as described in claim 62 and further comprising the step of enticing use of said internet-based recording system.

64. A method as described in claim 63 wherein said step of enticing use of said internet-based recording system comprises the step of tracking changes in test results for a user.

65. A method as described in claim 63 wherein said step of enticing use of said internet-based recording system comprises the steps of:
assessing potential error indicia; and
communicating potential errors to said user.

66. A method as described in claim 46 and further comprising the step of coordinating a supply quantity of said test with a supply quantity of said supplement.

67. A method as described in claim 46 and further comprising the step of instructing a user in a manner designed to reduce test results variability.

68. A method as described in claim 67 wherein said step of instructing a user in a manner designed to reduce test results variability comprises the step of directing said user to achieve an approximately constant parameter test procedure.

69. A method as described in claim 68 wherein said step of directing said user to achieve an approximately constant parameter test procedure comprises the step of directing test processes selected from a group comprising: a time-of-day based test procedure, a pre-meal test procedure, a meal type test procedure, a same-time-of-day test procedure, a morning based test procedure, an evening based test procedure, a twice-a-day test procedure, a three-time-a-day test procedure, a weekly test procedure, a one hour after meal test procedure, a three hours after meal test procedure, a menstrual cycle timed test procedure, and a specific meals tie test procedure.

70-72. (canceled)

73. A method as described in claim 18 and further comprising the step of making an on-site test available at a time of sale of said supplement.

74. A method as described in claim 18 and further comprising the step of making a send-in test available at a time of sale of said supplement.

75. A method as described in claim 18 and further comprising the step of making a supplement-need-indicative test available at a time of sale of said supplement.

76. A method as described in claim 18 and further comprising the step of making a supplement efficacy-based test available at a time of sale of said supplement.

77. A method as described in claim 18 and further comprising the step of making a user-profiled test available at a time of sale of said supplement.

78. A method as described in claim 77 wherein said step of making a user-profiled test available at a time of sale of said supplement makes available user-profiled test selected from a group comprising: a children-targeted test, a women-targeted test, a men-targeted test, a menstruational user-targeted test, an age targeted test, a prior history-targeted test, a symptomatic-targeted test, a coffee drinker-targeted test, and a prior blood test result-targeted test.

79. A method as described in claim 18 and further comprising the step of making an immediate results test available at a time of sale of said supplement.

80. A method as described in claim 79 wherein said immediate results test comprises a test yielding results in time selected from a group comprising within about three seconds, within about six seconds, and within about ten seconds.

81. A method as described in claim 18 and further comprising the step of making a test available at a time of sale of said supplement wherein said test yields results in a time selected from a group comprising within about twenty seconds, within about sixty seconds, and within about three hundred seconds.

82. A method as described in claim 18 and further comprising the step of making an internet reportable test available at a time of sale of said supplement.

83-104. (canceled)

105. A method as described in claim 18 wherein said step of selecting said test comprises the step of selecting a test from a group comprising: a qualitative test modality, a semi quantitative test modality, a quantitative test modality, a rate of change test modality, an absolute value test modality, a relative value test modality, and a fail safe test modality.

106. A method as described in claim 18 wherein said step of selecting said test comprises the step of selecting a test modality that is directly relevant to the use of said supplement.

107-109. (canceled)

110. A method as described in claim 18 wherein said step of selecting said test comprises the step of selecting a test modality that is indirectly relevant of the use of said supplement.

111. A method as described in claim 110 wherein said step of selecting a test modality that is indirectly relevant of the use of said supplement comprises the step of selecting a publicly espoused test modality.

112. A method as described in claim 110 wherein said step of selecting a test modality that is indirectly relevant of the use of said supplement comprises the step of selecting a popularly used test.

113. A method as described in claim 18 and further comprising the step of selecting a personal baseline test.

114-131. (canceled)

132. A system as described in claim 18 wherein said test comprises a test from a group comprising: a test strip-based test, a chip-based test, a culture-based test, an absorption-based test, a chromatagraphy-based test, an antibody-based test, a dye-based test, a blood thinner-based test, a vasodilator-based test, an AIDS-based test, a hormone-based test, a temperature-based test, a temperature strip-based test, a thermography strip test, a peripheral circulation-based test, a user extremity-based test, a red blood cell-based test, a blood presence-based test, an electrical conductivity-based test, a skin electrical conductivity-based test, a galvanic skin response-based test, a magnetic response-based test, a magnetic field-based test, an electrical field-based test, an electrical current-based test, a color coded results-based test, a testosterone-based test, an absorption-based test, a dipstick-type test, a vaginal fluid-based test, a sexually transmitted disease-based test, an enzyme linked immuno serum assay-based test, a kirlian photography-based test, a reaction time-based test, a photoelectric stimulus-based test, an alcohol presence-based test, a breath-based test, a blood-based test, an enzyme-based test, a virus-based test, a hormone-based test, a fertility-based test, a sperm motility-based test, a sperm count-based test, a viral byproduct-based test, a neuramidase-based test, a candida-based test, and a PCR-based test.

133. A system as described in claim 18 wherein said test comprises a saliva-sensitive test.

134. A system as described in claim 18 wherein said test comprises a test from a group comprising: a urine-based test, hair-based test, nail-based test, a non-invasive test, and a blood-based test.

135. A system as described in claim 18 wherein said step of selecting said test comprises the step of selecting a pH-based test.

136. A system as described in claim 18 wherein said test comprises a test from a group comprising: a ketone-based test, urea-based test, a serum albumin-based test, a hormone-based test, an immunoassay, an enzymatic assay, a free radical-based test, a redox-based test, an oxidative metabolite-based test, an IgG-based test, an IgA-based test, an IgM-based test, a venous plasma pH-based test, an arterial pH-based test, a free radical-based test, an antioxidant-based test, a chemical reaction-based test, an NPN-based test, and a PNP-based test.

137-189. (canceled)
